(19) United States
(12) Patent Application Publication
(10) Pub. No.: US 2023/0248422 A1
(43) Pub. Date: Aug. 10, 2023

(54) THERMAL ABLATION DEVICE

(71) Applicant: MINERVA SURGICAL, INC., Santa Clara, CA (US)

(72) Inventors: Estela Hilario, Los Altos, CA (US); Sean Darby, Gilroy, CA (US)

(73) Assignee: MINERVA SURGICAL, INC., Santa Clara, CA (US)

(21) Appl. No.: 18/163,687

(22) Filed: Feb. 2, 2023

Related U.S. Application Data
(60) Provisional application No. 63/308,688, filed on Feb. 10, 2022.

(51) Int. Cl. A61B 18/14

(52) U.S. Cl. CPC A61B 18/1485; A61B 2018/00559

(57) ABSTRACT

A thermal ablation device includes proximal and distal portions, a connector disposed therebetween, and an elongate shaft extending within the proximal portion, the connector, and the distal portion. The distal portion includes a distal inner tube disposed radially outward of the elongate shaft and fixedly attached to the connector, a distal outer tube disposed radially outward of the distal inner tube, a distal tip member fixedly attached to distal ends of the distal inner tube, the distal outer tube, and the elongate shaft, a sealing sleeve fixedly attached to an outer surface of the distal outer tube, the sealing sleeve including a plurality of deflectable fins extending radially outward from the sealing sleeve, and a sealing balloon fixedly attached to the distal outer tube at a proximal waist distal of the plurality of deflectable fins and at a distal waist proximal of the distal tip member.

### CROSS REFERENCE TO RELATED APPLICATIONS

This application claims the benefit of U.S. Provisional Patent Application Ser. No. 63/308,688 filed on Feb. 10, 2022, the disclosure of which is incorporated herein by reference.

### FIELD

The present disclosure relates generally to thermal ablation devices. More particularly, the present disclosure relates to thermal ablation devices including a sealing balloon.

### BACKGROUND

Uterine bleeding may arise from hormonal imbalances, system disease, or anatomical abnormalities such as fibroids, polyps, or other growths. A common non-invasive treatment for such conditions may include hydro-thermal ablation (HTA), where heated fluid (e.g., saline solution) is supplied to the uterus via a device inserted into through the cervical canal to ablate the endometrial lining of the uterus. However, leakage of the heated fluid from the uterus may damage non-targeted tissue. There is an ongoing need for alternative tissue ablation devices and/or methods of use and/or manufacture of said devices.

### SUMMARY

In one example, a thermal ablation device may comprise a proximal portion; a distal portion sized and configured for insertion through a cervical opening; a connector disposed between the proximal portion and the distal portion; and an elongate shaft extending within the proximal portion, the connector, and the distal portion. The elongate shaft may define a working lumen. The distal portion may include a distal inner tube disposed radially outward of the elongate shaft and fixedly attached to the connector, a distal outer tube disposed radially outward of the distal inner tube, a distal tip member fixedly attached to a distal end of the distal inner tube, a distal end of the distal outer tube, and a distal end of the elongate shaft, a sealing sleeve fixedly attached to an outer surface of the distal outer tube, the sealing sleeve including a plurality of deflectable fins extending radially outward from the sealing sleeve, and a sealing balloon configured to shift between a deflated configuration and an inflated configuration, the sealing balloon being fixedly attached to the distal outer tube at a proximal waist distal of the plurality of deflectable fins and at a distal waist proximal of the distal tip member.

In addition or alternatively to any example described herein, the sealing balloon includes a first cuff, a second cuff, and a central portion extending from the first cuff to the second cuff in the deflated configuration.

In addition or alternatively to any example described herein, the second cuff is spaced axially apart from the first cuff in the deflated configuration by the central portion of the sealing balloon.

In addition or alternatively to any example described herein, the first cuff defines a generally annular shape in the deflated configuration.

In addition or alternatively to any example described herein, the second cuff defines a generally annular shape in the deflated configuration.

In addition or alternatively to any example described herein, in the inflated configuration of the sealing balloon, the central portion of the sealing balloon defines a radially outermost extent of the sealing balloon.

In addition or alternatively to any example described herein, in the deflated configuration of the sealing balloon, the first cuff and the second cuff each extend radially outward a greater distance from the distal outer tube than the central portion of the sealing balloon.

In addition or alternatively to any example described herein, the proximal portion includes a proximal inner tube disposed radially outward of the elongate shaft and fixedly attached to the connector, a proximal middle tube disposed radially outward of the proximal inner tube and spaced apart proximally from the connector, and a proximal outer tube disposed radially outward of the proximal middle tube and fixedly attached to the connector.

In addition or alternatively to any example described herein, the thermal ablation device may include an inflation lumen in fluid communication with the sealing balloon, wherein at least a portion of the inflation lumen is defined by the proximal middle tube and the proximal outer tube.

In addition or alternatively to any example described herein, at least a portion of the inflation lumen is defined by a recess formed in a wall of the proximal outer tube or a recess formed in a wall of the proximal middle tube.

In addition or alternatively to any example described herein, an outer surface of the proximal middle tube is in contact with an inner surface of the proximal outer tube.

In addition or alternatively to any example described herein, the proximal middle tube is radially spaced apart from the proximal inner tube by a first distance and the proximal middle tube is radially spaced apart from the proximal outer tube by a second distance.

In addition or alternatively to any example described herein, the proximal portion further includes a first O-ring disposed between the proximal inner tube and the proximal middle tube, and a second O-ring disposed between the proximal inner tube and the proximal middle tube. The proximal inner tube, the first O-ring, the second O-ring, and the proximal middle tube define an insulating air gap extending along a majority of the proximal portion.

In addition or alternatively to any example described herein, the first distance is greater than the second distance.

In addition or alternatively to any example described herein, a thermal ablation device may comprise a proximal portion including a handpiece configured to connect to a fluid management system and an inflation port disposed distal of the handpiece; a distal portion sized and configured for insertion through a cervical opening; a connector fixedly securing the proximal portion to the distal portion; and an elongate shaft extending within the proximal portion, through the connector, and within the distal portion. The elongate shaft may define a working lumen. The distal portion may include a distal inner tube disposed radially outward of the elongate shaft and fixedly attached to the connector, a distal outer tube disposed radially outward of the distal inner tube, a distal tip member fixedly attached to a distal end of the distal inner tube, a distal end of the distal outer tube, and a distal end of the elongate shaft, a sealing sleeve fixedly attached to an outer surface of the distal outer tube, the sealing sleeve including a plurality of deflectable fins extending radially outward from the sealing sleeve, and a sealing balloon in fluid communication with the inflation port and configured to shift between a deflated configuration and an inflated configuration, the sealing balloon being fixedly attached to the distal outer tube at a proximal waist distal of the plurality of deflectable fins and at a distal waist proximal of the distal tip member.

In addition or alternatively to any example described herein, the sealing balloon is monolithically formed with the plurality of deflectable fins.

In addition or alternatively to any example described herein, the sealing balloon is fixedly attached to the distal outer tube independently of the plurality of deflectable fins.

In addition or alternatively to any example described herein, the sealing balloon has a fluid capacity of less than 5 milliliters of inflation fluid.

In addition or alternatively to any example described herein, in the deflated configuration, the sealing balloon defines a first chamber and a second chamber spaced apart from the first chamber. In the inflated configuration, the sealing balloon defines a single interior chamber.

In addition or alternatively to any example described herein, a thermal ablation device may comprise a proximal portion having a first outer diameter; a distal portion having a second outer diameter less than the first outer diameter; and a connector disposed between the proximal portion and the distal portion. An outer surface of the connector is tapered from the first outer diameter to the second outer diameter. The distal portion includes a distal inner tube fixedly attached to the connector and a distal tip member, a distal outer tube disposed radially outward of the distal inner tube and fixedly attached to the connector and the distal tip member, a sealing sleeve fixedly attached to an outer surface of the distal outer tube, the sealing sleeve including a plurality of deflectable fins monolithically formed therewith and extending radially outward from the sealing sleeve, and a sealing balloon configured to shift between a deflated configuration and an inflated configuration, the sealing balloon being fixedly attached to the distal outer tube at a proximal waist distal of the plurality of deflectable fins and at a distal waist proximal of the distal tip member.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view illustrating selected aspects of a thermal ablation device;

FIG. 2 is a side cross-sectional view illustrating selected aspects of a proximal portion of the thermal ablation device of FIG. 1;

FIG. 3 is a perspective view illustrating selected aspects of a distal portion of the thermal ablation device of FIG. 1;

FIGS. 4-5 are side cross-sectional views illustrating selected aspects of the distal portion of the thermal ablation device of FIG. 1;

FIG. 6 is a cross-sectional view illustrating selected aspects of the proximal portion of the thermal ablation device of FIG. 1;

FIG. 7 is a cross-sectional view illustrating selected aspects of the distal portion of the thermal ablation device of FIG. 1;

FIG. 8 is a cross-sectional view illustrating selected aspects of the proximal portion of an alternative embodiment of the thermal ablation device of FIG. 1;

FIG. 9 is a cross-sectional view illustrating selected aspects of the proximal portion of an alternative embodiment of the thermal ablation device of FIG. 1; and

FIGS. 10-11 schematically illustrate selected aspects of the thermal ablation device of FIG. 1 during use.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to some features may be equally referred to all instances and quantities beyond one of said feature(s) unless clearly stated to the contrary. As such, it will be understood that the following discussion may apply equally to any and/or all of the components for which there are more than one within the device, etc. unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

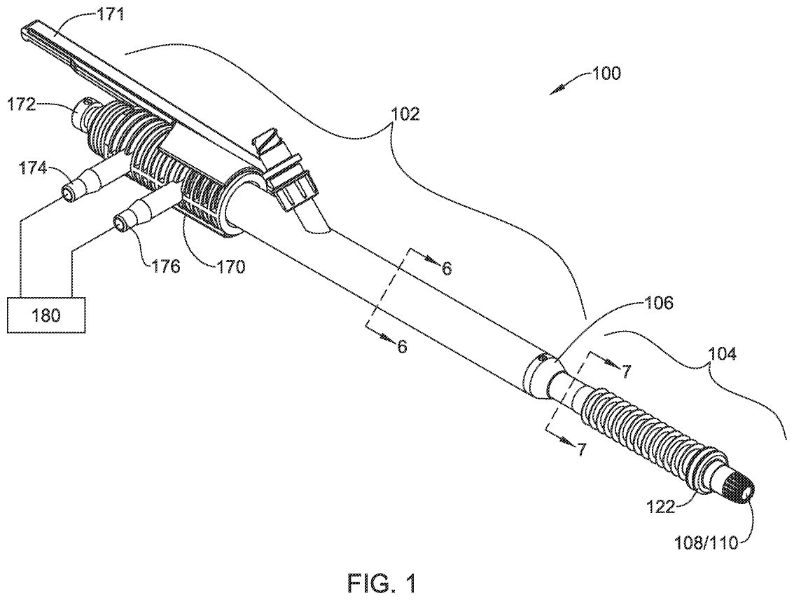

FIG. 1 is a perspective view illustrating selected aspects of a thermal ablation device 100. The thermal ablation device 100 may include a proximal portion 102 and a distal portion 104. In at least some embodiments, the distal portion 104 may be sized and configured for insertion through a cervical opening. The proximal portion 102 may have a first outer diameter. The distal portion 104 may have a second outer diameter less than the first outer diameter.

The thermal ablation device 100 may include a connector 106 disposed between the proximal portion 102 and the distal portion 104. In some embodiments, the connector 106 may fixedly secure and/or may fixedly attached the proximal portion 102 to the distal portion 104. In at least some embodiments, an outer surface of the connector 106 may be tapered from the first outer diameter at and/or adjacent a proximal end of the connector 106 to the second outer diameter at and/or adjacent a distal end of the connector 106. Some suitable but non-limiting examples of materials that may be used to form the connector 106, including but not limited to polymers, metals, composites, and the like, are described below. In one example, the connector 106 may be formed from a polymeric material.

The thermal ablation device 100 may include an elongate shaft 108 disposed and/or extending within the proximal portion 102, the connector 106, and the distal portion 104. In at least some embodiments, the elongate shaft 108 may extend within the proximal portion 102, through the connector 106, and within the distal portion 104. In some embodiments, the elongate shaft 108 may extend from within the proximal portion 102 to within the distal portion 104. The elongate shaft 108 may define a working lumen 110 configured to receive an endoscopic device and/or other medical device(s) as necessary for a particular procedure. In some embodiments, the working lumen 110 of the elongate shaft 108 may be in communication with a proximal access port 172, as discussed herein. Some suitable but non-limiting examples of materials that may be used to form the elongate shaft 108, including but not limited to polymers, metals, composites, and the like, are described below. In one example, the elongate shaft 108 may be formed from a metallic material.

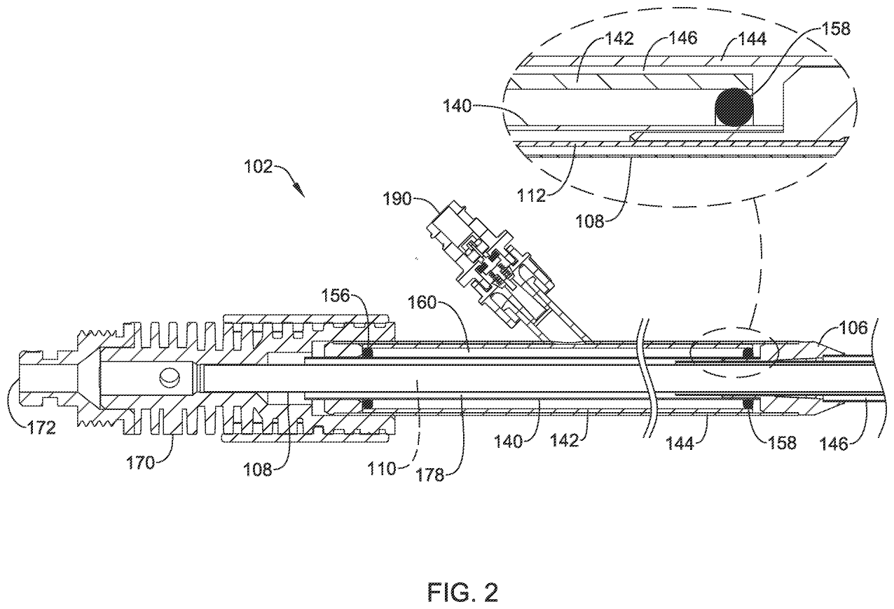

The proximal portion 102 of the thermal ablation device 100 may include a handpiece 170 configured to connect to a fluid management system 180. The proximal portion 102 and/or the handpiece 170 may include the proximal access port 172 in fluid communication with the working lumen 110 of the elongate shaft 108. In some embodiments, the proximal access port 172 may be configured to receive an endoscopic device therein and/or may be configured to direct the endoscopic device into the working lumen 110 of the elongate shaft 108. The proximal portion 102 and/or the handpiece 170 may include an irrigation port 174 and/or an aspiration port 176. The irrigation port 174 may be in fluid communication with the working lumen 110 of the elongate shaft 108. The irrigation port 174 may be configured to be fluidly connected to the fluid management system 180 and/or a source of ablation fluid. The aspiration port 176 may be in fluid communication with an aspiration lumen 178 (e.g., FIG. 2), as described herein. The aspiration port 176 may be configured to be fluidly connected to the fluid management system 180 and/or a source of suction.

In some embodiments, the proximal portion 102 may optionally include a tenaculum stabilizer 171 on and/or coupled to the handpiece 170. During an HTA procedure, a tenaculum (not shown) may be placed around the cervical canal and coupled to the thermal ablation device 100 to help hold the thermal ablation device 100 at a desired position and depth within the uterus and/or the cervical canal. In some embodiments, the tenaculum stabilizer 171 may aid in preventing the thermal ablation device 100 from being withdrawn proximally from the uterus and/or the cervical canal during the procedure to avoid exposing non-targeted tissue to the ablation fluid.

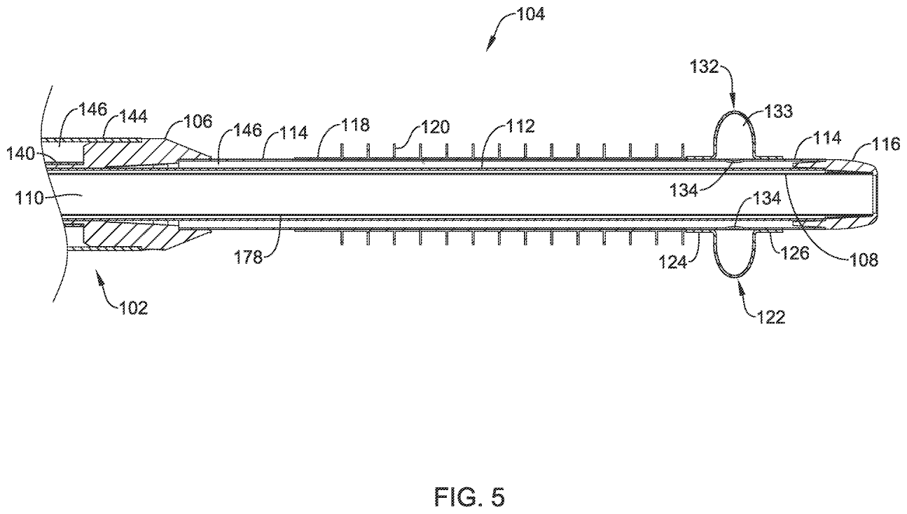

In some embodiments, the distal portion 104 may include a sealing balloon 122 configured to shift between a deflated configuration (e.g., FIGS. 1, 3-4) and an inflated configuration (e.g., FIG. 5). The sealing balloon 122 will be described in more detail below. For reference, the sealing balloon 122 may be disposed proximate a distal end of the elongate shaft 108 and/or a distal end of the distal portion 104.

As may be seen in FIG. 2, the proximal portion 102 may include a proximal inner tube 140 disposed radially outward of the elongate shaft 108. The proximal inner tube 140 may be fixedly attached to the connector 106. The proximal inner tube 140 may be radially spaced apart from the elongate shaft 108. In some embodiments, the proximal inner tube 140 may extend into the handpiece 170. In some embodiments, the proximal inner tube 140 may terminate within the handpiece 170. In some embodiments, the proximal inner tube 140 may be fixedly attached to the handpiece 170. In some embodiments, a proximal portion of the aspiration lumen 178 may be at least partially defined by an outer surface of the elongate shaft 108 and an inner surface of the proximal inner tube 140.

In some embodiments, the proximal portion 102 may include a proximal middle tube 142 disposed radially outward of the proximal inner tube 140. The proximal middle tube 142 may be axially spaced apart proximally from the connector 106. The proximal middle tube 142 may be radially spaced apart from the proximal inner tube 140. In at least some embodiments, the proximal middle tube 142 may be secured to and/or may be fixedly attached to the handpiece 170. The proximal middle tube 142 may have a length that is less than a length of the proximal inner tube 140.

In some embodiments, the proximal portion 102 may include a proximal outer tube 144 disposed radially outward of the proximal middle tube 142. The proximal outer tube 144 may be fixedly attached to the connector 106. The proximal outer tube 144 may have a length that is greater than the length of the proximal middle tube 142.

In some embodiments, the thermal ablation device 100 and/or the proximal portion 102 may include an inflation port 190 disposed distal of the handpiece 170. In some embodiments, the inflation port 190 may include a valve disposed therein. In some embodiments, the valve may be selectively actuated between an open position and a closed position. In some embodiments, the valve may be in a normally closed configuration. In some embodiments, the valve may be opened by a feature extending distally from a connector used to connect to the inflation port 190. In one example, a syringe may be used to supply inflation fluid to the inflation port 190. The syringe may include a feature such as a protrusion extending distally therefrom that engages the valve and pushes the valve from the closed position to the open position when the syringe is connected to the inflation port 190. Other configurations are also contemplated. In some embodiments, the syringe may include a locking feature configured to prevent withdrawal of the inflation fluid after the inflation fluid has been injected into the inflation port 190.

In some embodiments, the thermal ablation device 100 may include an inflation lumen 146 in fluid communication with the sealing balloon 122 (e.g., FIGS. 1, 4-5). In some embodiments, the inflation lumen 146 may extend from the inflation port 190, within the proximal portion 102, through the connector 106, and within the distal portion 104 to the sealing balloon 122. Thus, the inflation port 190 may be in fluid communication with the interior of the sealing balloon 122 to provide inflation fluid thereto.

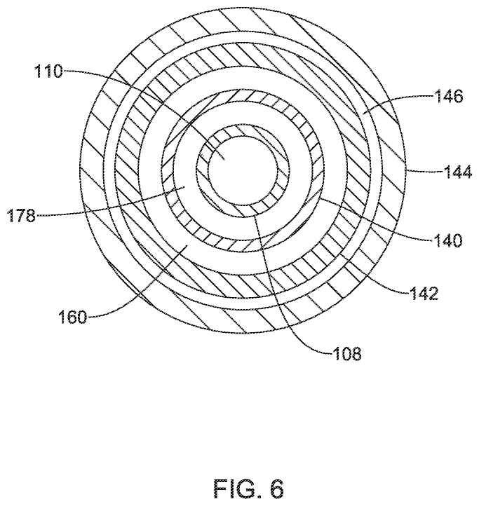

In some embodiments, at least a portion of the inflation lumen 146 may be defined by the proximal middle tube 142 and the proximal outer tube 144. In some embodiments, at least a portion of the inflation lumen 146 may be defined between the proximal middle tube 142 and the proximal outer tube 144, such as by an outer surface of the proximal middle tube 142 and an inner surface of the proximal outer tube 144. In some embodiments, the proximal outer tube 144 may be radially spaced apart from the proximal middle tube 142 (e.g., FIGS. 2, 6). In some embodiments, the proximal middle tube 142 may be radially spaced apart from the proximal inner tube 140 by a first distance. In some embodiments, the proximal middle tube 142 may be radially spaced apart from the proximal outer tube 144 by a second distance. In at least some embodiments, the second distance may be different from the first distance. In some embodiments, the first distance may be greater than the second distance. Other configurations are also contemplated.

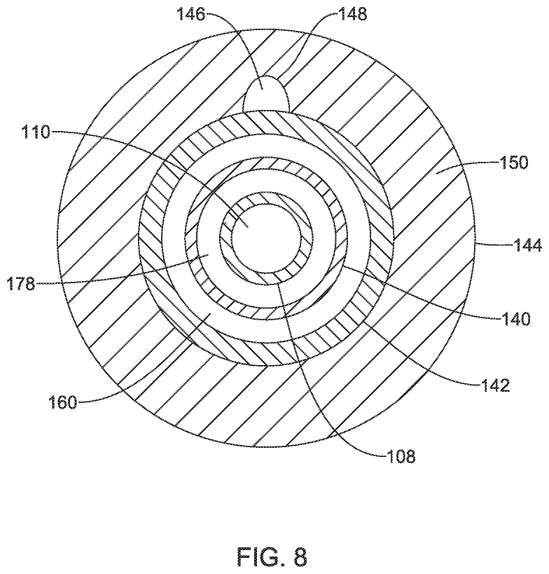

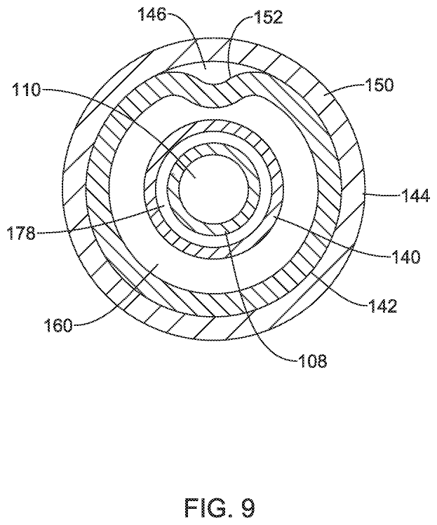

In some alternative embodiments, at least a portion of the inflation lumen 146 may be defined by a recess 148 formed in a wall 150 of the proximal outer tube 144, as seen in FIG. 8, or a recess 152 formed in a wall 154 of the proximal middle tube 142, as seen in FIG. 9. In some embodiments, the outer surface of the proximal middle tube 142 may be in contact with the inner surface of the proximal outer tube 144. Other configurations are also contemplated.

Returning to FIG. 2, in some embodiments, the proximal portion 102 may include a first O-ring 156 disposed between the proximal inner tube 140 and the proximal middle tube 142. The first O-ring 156 may be in sealing engagement with an outer surface of the proximal inner tube 140 and an inner surface of the proximal middle tube 142. The first O-ring 156 may be disposed at and/or proximate a proximal end of the proximal middle tube 142. In some embodiments, the proximal portion 102 may include a second O-ring 158 disposed between the proximal inner tube 140 and the proximal middle tube 142. The second O-ring 158 may be in sealing engagement with the outer surface of the proximal inner tube 140 and the inner surface of the proximal middle tube 142. The second O-ring 158 may be disposed at and/or proximate a distal end of the proximal middle tube 142. In at least some embodiments, the proximal inner tube 140, the first O-ring 156, the second O-ring 158, and the proximal middle tube 142 may define an insulating air gap 160 extending along a majority of the proximal portion 102. The insulating air gap 160 may prevent or limit heat transfer from the ablation fluid passing through the working lumen 110 of the elongate shaft 108 through the proximal portion 102 and/or the proximal outer tube 144, thereby protecting non-targeted tissue from heat damage.

As may be seen in FIG. 2, the insulating air gap 160 (as defined by the proximal inner tube 140, the first O-ring 156, the second O-ring 158, and the proximal middle tube 142) may also serve to reduce the cross-sectional area and/or the volume of the inflation lumen 146, or of the at least a portion of the inflation lumen 146, disposed and/or extending within the proximal portion 102. Since some inflation fluids are compressible, a greater volume of inflation fluid may be required to overcome the compressibility of the inflation fluid when shifting the sealing balloon 122 from the deflated configuration to the inflated configuration depending upon the volume of the inflation lumen 146. By reducing the volume of the inflation lumen 146, less inflation fluid is required to inflate the sealing balloon 122 and greater control over inflation of the sealing balloon 122 may be obtained.

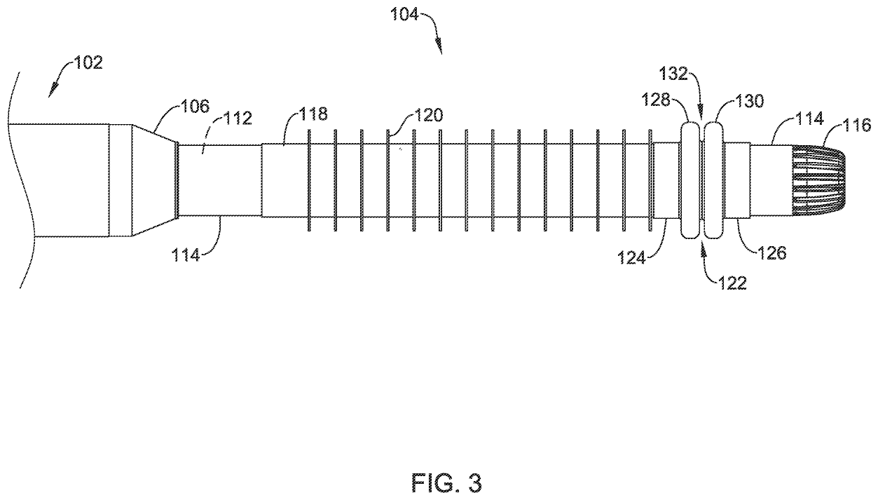

Turning now to FIGS. 3-5, in some embodiments, the distal portion 104 may include a distal inner tube 112 disposed radially outward of the elongate shaft 108 and fixedly attached to the connector 106. In some embodiments, the distal inner tube 112 may extend through the connector 106. In some embodiments, the distal inner tube 112 may extend proximal of the connector 106. Some suitable but non-limiting examples of materials that may be used to form the distal inner tube 112, including but not limited to polymers, metals, composites, and the like, are described below. In one example, the distal inner tube 112 may be formed from a polymeric material.

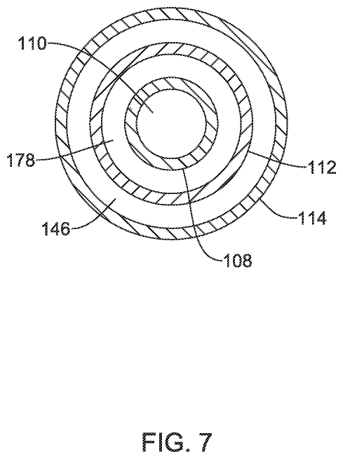

In some embodiments, the distal portion 104 may include a distal outer tube 114 disposed radially outward of the distal inner tube 112 and fixedly attached to the connector 106. In some embodiments, the distal outer tube 114 may be disposed coaxially with the distal inner tube 112 (e.g., FIG. 7). Some suitable but non-limiting examples of materials that may be used to form the distal outer tube 114, including but not limited to polymers, metals, composites, and the like, are described below. In one example, the distal outer tube 114 may be formed from a polymeric material.

In some embodiments, the distal portion 104 may include a distal tip member 116 fixedly attached to a distal end of the distal inner tube 112, a distal end of the distal outer tube 114, and/or the distal end of the elongate shaft 108. In some embodiments, the distal tip member 116 may extend distal of the elongate shaft 108, the distal inner tube 112, and/or the distal outer tube 114. In at least some embodiments, the distal tip member 116 may be disposed at and/or may extend to a distalmost end of the distal portion 104 and/or the thermal ablation device 100. In some embodiments, the distal tip member 116 may include one or more channels formed therein, wherein the one or more channels is in fluid communication with the aspiration lumen 178. In some embodiments, a distal portion of the aspiration lumen 178 may be at least partially defined by the outer surface of the elongate shaft 108 and an inner surface of the distal inner tube 112. In some embodiment, a distal portion of the aspiration lumen 178 may be at least partially defined between the elongate shaft 108 and the distal inner tube 112. In some embodiments, the connector 106 may include at least one fluid channel fluidly coupling the proximal portion of the aspiration lumen 178 and the distal portion of the aspiration lumen 178. Other configurations are also contemplated. Some suitable but non-limiting examples of materials that may be used to form the distal tip member 116, including but not limited to polymers, metals, composites, and the like, are described below. In one example, the distal tip member 116 may be formed from a polymeric material.

In some embodiments, the distal portion 104 may include a sealing sleeve 118 fixedly attached to an outer surface of the distal outer tube 114. In some embodiments, the sealing sleeve 118 may be bonded directly to the distal outer tube 114. In some embodiments, the sealing sleeve 118 may be overmolded onto the distal outer tube 114. Other configurations are also contemplated. In some embodiments, the sealing sleeve 118 may include a plurality of deflectable fins 120 extending radially outward from the sealing sleeve 118 and/or the distal outer tube 114. In at least some embodiments, each of the plurality of deflectable fins 120 may have an axial thickness that is less than its radial extent from the sealing sleeve 118. In some embodiments, the plurality of deflectable fins 120 may be monolithically formed with the sealing sleeve 118 as a single unitary structure. Some suitable but non-limiting examples of materials that may be used to form the sealing sleeve 118 and/or the plurality of deflectable fins 120, including but not limited to polymers, composites, and the like, are described below. In some embodiments, the sealing sleeve 118 and/or the plurality of deflectable fins 120 may be formed from a polymeric material that is softer and/or more flexible than the distal outer tube 114. In one example, the sealing sleeve 118 and/or the plurality of deflectable fins 120 may be formed from silicone. Other configurations and/or materials are also contemplated.

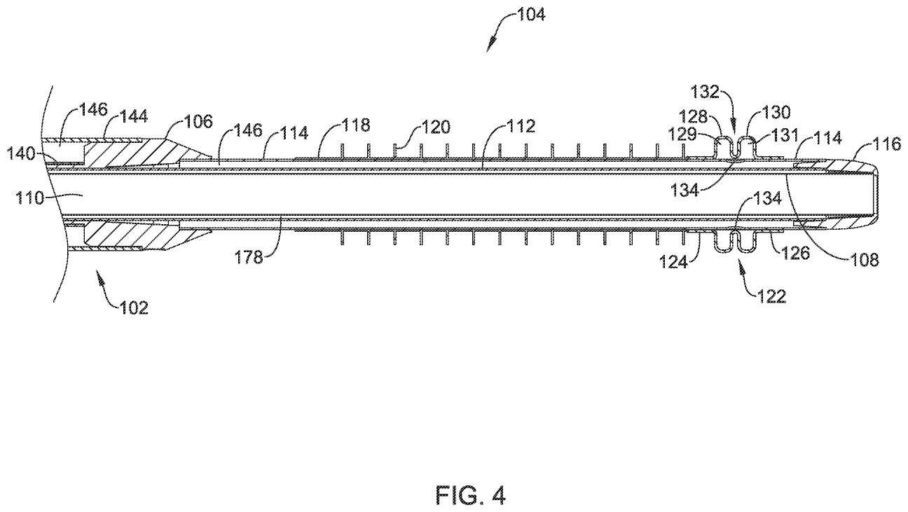

In some embodiments, the distal portion 104 may include the sealing balloon 122 configured to shift between the deflated configuration (e.g., FIG. 4) and the inflated configuration (e.g., FIG. 5). The sealing balloon 122 may be fixedly attached to the distal outer tube 114 at a proximal waist 124 distal of the plurality of deflectable fins 120 and at a distal waist 126 proximal of the distal tip member 116. In some embodiments, the sealing balloon 122 may be formed separately and/or independently of the sealing sleeve 118 and/or the plurality of deflectable fins 120. In some embodiments, the sealing balloon 122 may be fixedly attached to the distal outer tube 114 independently of the sealing sleeve 118 and/or the plurality of deflectable fins 120. In some embodiments, the sealing balloon 122, and/or the proximal waist 124 and the distal waist 126 of the sealing balloon 122, may be adhesively bonded to the distal outer tube 114. In some embodiments, the sealing balloon 122 may be monolithically formed with the sealing sleeve 118 and/or the plurality of deflectable fins 120 as a single unitary structure.

In some embodiments, the sealing balloon 122 may include a first cuff 128, a second cuff 130, and a central portion 132 extending circumferentially around the sealing balloon 122 between the first cuff 128 to the second cuff 130 in the deflated configuration. In at least some embodiments, the second cuff 130 may be axially spaced apart from the first cuff 128 in the deflated configuration. In some embodiments, the second cuff 130 may be axially spaced apart from the first cuff 128 in the deflated configuration by the central portion 132 of the sealing balloon 122.

In some embodiments, the first cuff 128 may define a generally annular shape in the deflated configuration. In some embodiments, the first cuff 128 may define a first chamber 129 of the sealing balloon 122 in the deflated configuration, as seen in FIG. 4. The first cuff 128 may be configured to maintain its shape in the deflated configuration, even if the first chamber 129 is devoid of inflation fluid. In some embodiments, the second cuff 130 may define a generally annular shape in the deflated configuration. In some embodiments, the second cuff 130 may define a second chamber 131 of the sealing balloon 122 in the deflated configuration, as seen in FIG. 4. The second cuff 130 may be configured to maintain its shape in the deflated configuration, even if the second chamber 131 is devoid of inflation fluid. As such, in the deflated configuration, the sealing balloon 122 may define the first chamber 129 and the second chamber 131, wherein the second chamber 131 is spaced apart from the first chamber 129.

In at least some embodiments, in the deflated configuration of the sealing balloon 122, the first cuff 128 and/or the second cuff 130 may each extend radially outward a greater distance from the distal outer tube 114 than the central portion 132 of the sealing balloon 122. Thus, the central portion 132 may be an annular recessed portion of the sealing balloon 122 between the first cuff 128 and the second cuff 130 in the deflated configuration. In at least some embodiments, in the inflated configuration of the sealing balloon 122, the central portion 132 of the sealing balloon 122 may define a radially outermost extent of the sealing balloon 122, as seen in FIG. 5. In some embodiments, in the inflated configuration, the sealing balloon 122 may define a single interior chamber 133. Accordingly, the first chamber 129 and the second chamber 131 may merge to become the single interior chamber 133 as the sealing balloon 122 shifts from the deflated configuration to the inflated configuration.

In some embodiments, the sealing balloon 122 may have a fluid capacity of 10 milliliters or less of inflation fluid in the inflated configuration. In some embodiments, the sealing balloon 122 may have a fluid capacity of 7.5 milliliters or less of inflation fluid in the inflated configuration. In some embodiments, the sealing balloon 122 may have a fluid capacity of 5 milliliters or less of inflation fluid in the inflated configuration. Other configurations are also contemplated. The sealing balloon 122 may be inflated with any desired inflation fluid. In some embodiments, the inflation fluid may be air or another biocompatible gas. In some embodiments, the inflation fluid may be saline solution or another biocompatible liquid. Other inflation fluids are also contemplated.

In some embodiments, the distal portion 104 and/or the distal outer tube 114 may include at least one inflation aperture 134 formed in and/or extending through the distal outer tube 114. Each aperture of the at least one inflation aperture 134 may be in fluid communication with both the first cuff 128 and the second cuff 130. Each aperture of the at least one inflation aperture 134 may be in fluid communication with both the first chamber 129 and the second chamber 131. Other configurations are also contemplated. For example, in some embodiments, an equal number of inflation apertures may be in fluid communication with each of the first cuff 128 and the second cuff 130 (and/or the first chamber 129 and the second chamber 131) such that the first cuff 128 and the second cuff 130 (and/or the first chamber 129 and the second chamber 131) inflate equally and/or at the same time. In some embodiments, an unequal number of inflation apertures may be in fluid communication with each of the first cuff 128 and the second cuff 130 (and/or the first chamber 129 and the second chamber 131) such that the first cuff 128 and the second cuff 130 (and/or the first chamber 129 and the second chamber 131) inflate sequentially and/or in an unequal manner.

In some embodiments, at least a portion of the inflation lumen 146 may be defined by the distal inner tube 112 and the distal outer tube 114. In some embodiments, at least a portion of the inflation lumen 146 may be defined by an outer surface of the distal inner tube 112 and an inner surface of the distal outer tube 114. In some embodiments, the distal outer tube 114 may be radially spaced apart from the distal inner tube 112 (e.g., FIGS. 4, 7). In some embodiments, the inflation lumen 146 may be defined between the distal outer tube 114 and the distal inner tube 112. Other configurations are also contemplated. In some embodiments, at least a portion of the inflation lumen 146 may be defined by the distal inner tube 112, the distal outer tube 114, and the distal tip member 116. In some embodiments, the distal tip member 116 may close off and/or may define a distal end of the inflation lumen 146. The inflation lumen 146 may be in fluid communication with the at least one inflation aperture 134.

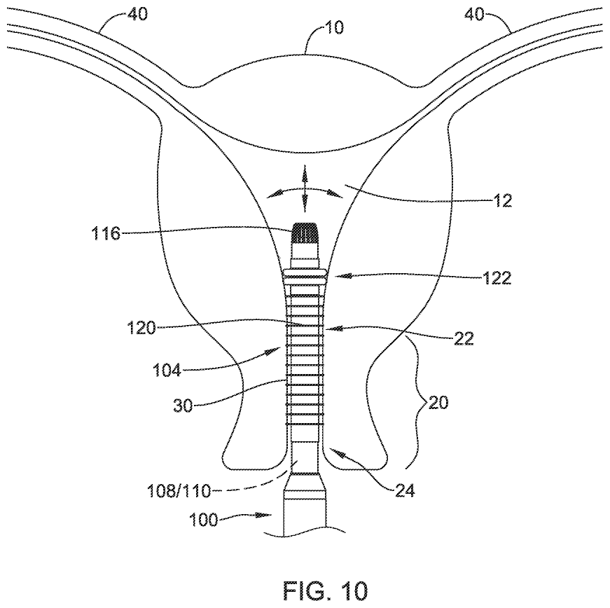

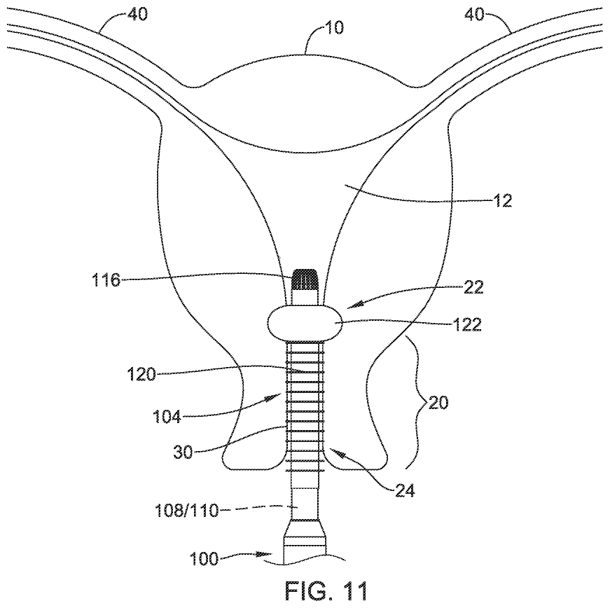

FIGS. 10 and 11 illustrate using the thermal ablation device 100 within a uterus 10 defining a uterine cavity 12. The uterus 10 is connected to the cervix 20, which defines an entrance to the uterus 10 and/or the uterine cavity 12. The cervix 20 includes an inner os 22 (e.g., an inner orifice) and an outer os 24 (e.g., an outer orifice). The cervix 20 includes and/or defines the cervical canal 30, which extends from the outer os 24 to the inner os 22. Fallopian tubes 40, which connect to the ovaries (not shown), are connected to the uterus 10 and are in communication with the uterine cavity 12.

When the distal portion 104 of the thermal ablation device 100 is inserted into the cervix 20 and/or the cervical canal 30 and advanced to the uterine cavity 12, the plurality of deflectable fins 120 may engage with the cervical canal 30 to form a passive seal, as seen in FIG. 10. The distal portion 104 of the thermal ablation device 100 may be movable within and/or relative to the cervical canal 30 to permit the thermal ablation device 100 and/or an endoscopic device extending therethrough to move within and/or to examine the uterine cavity 12. The sealing balloon 122 may be maintained in the deflated configuration during this process, thereby permitting the distal portion 104 of the thermal ablation device 100 to move within, through, and/or relative to the cervical canal 30.

In order to commence an HTA procedure, the distal portion 104 of the thermal ablation device 100 may be withdrawn from the uterine cavity 12 slightly until the sealing balloon 122 is positioned generally within the inner os 22 of the cervix 20. The plurality of deflectable fins 120 may continue to maintain a passive seal within the cervical canal 30. However, under pressure, the heated fluid introduced into the uterine cavity 12 through the working lumen 110 may distend the uterine cavity 12 and create opportunity for leakage through the cervical canal 30. To prevent this, the sealing balloon 122 may be shifted from the deflated configuration to the inflated configuration within the inner os 22 of the cervix 20, as shown in FIG. 11. The sealing balloon 122 may serve one or more purposes in the inflated configuration within the inner os 22. In some embodiments, the sealing balloon 122 may actively seal the cervical canal 30 in the inflated configuration, thereby adding another layer of sealing to prevent the heated fluid from contacting non-targeted tissue. In some embodiments, the sealing balloon 122 may anchor the distal portion 104 within and/or relative to the cervix 20 and/or the cervical canal 30. Other benefits and/or configurations are also contemplated.

After inflating the sealing balloon 122 to the inflated configuration, heated ablation fluid may be introduced into the uterine cavity 12 through the working lumen 110 or the elongate shaft 108. As the heated ablation fluid circulates within the uterine cavity 12, the heated ablation fluid may thermally ablate endometrial tissue exposed within the uterine cavity 12. The uterine cavity 12 may be distended by a certain amount but has a distension limit. As the distension limit is approached, some ablation fluid must be removed from the uterine cavity 12 before and/or while new, heated ablation fluid is introduced to the uterine cavity 12. The ablation fluid may also slowly cool as it circulates within the uterine cavity 12. The ablation fluid within the uterine cavity 12 may be aspirated through the one or more channels formed in the distal tip member 116 to the aspiration lumen 178 (e.g., FIGS. 2, 5) and back to the fluid management system 180 (e.g., FIG. 1) for filtering, re-heating, re-circulating, and/or disposal. As such, the distal tip member 116 may remain within the uterine cavity 12 distal of the inner os 22 such that the one or more channels formed in the distal tip member 116 remain unobstructed by tissue.

The materials that can be used for the various components of the thermal ablation device and the various elements thereof disclosed herein may include those commonly associated with medical devices. For simplicity purposes, the following discussion refers to the device. However, this is not intended to limit the devices, components, and methods described herein, as the discussion may be applied to other elements, members, components, or devices disclosed herein, such as, but not limited to, the proximal portion, the distal portion, the connector, the elongate shaft, the distal inner tube, the distal outer tube, the sealing sleeve, the sealing balloon, the proximal inner tube, the proximal middle tube, the proximal outer tube, the handpiece, the tenaculum stabilizer, etc. and/or elements or components thereof.

In some embodiments, the device and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM), polyether block ester, polyurethane, polypropylene (PP), polyvinylchloride (PVC), polyether-ester, ether or ester based copolymers (for example, butylene/poly(alkylene ether) phthalate and/or other polyester elastomers), polyamide, elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene, polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide, polysulfone, nylon, nylon-12, perfluoro(propyl vinyl ether) (PFA), ethylene vinyl alcohol, polyolefin, polystyrene, epoxy, polyvinylidene chloride (PVdC), poly(styrene-b-isobutylene-b-styrene), polycarbonates, polyisobutylene (PIB), polyisobutylene polyurethane (PIBU), polyurethane silicone copolymers, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; or any other suitable material.

In at least some embodiments, portions or all of the device and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the device in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the device to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed:

1. A thermal ablation device, comprising:
a proximal portion;
a distal portion sized and configured for insertion through a cervical opening;
a connector disposed between the proximal portion and the distal portion; and
an elongate shaft extending within the proximal portion, the connector, and the distal portion, the elongate shaft defining a working lumen;
wherein the distal portion includes:
a distal inner tube disposed radially outward of the elongate shaft and fixedly attached to the connector,
a distal outer tube disposed radially outward of the distal inner tube,
a distal tip member fixedly attached to a distal end of the distal inner tube, a distal end of the distal outer tube, and a distal end of the elongate shaft,
a sealing sleeve fixedly attached to an outer surface of the distal outer tube, the sealing sleeve including a plurality of deflectable fins extending radially outward from the sealing sleeve, and
a sealing balloon configured to shift between a deflated configuration and an inflated configuration, the sealing balloon being fixedly attached to the distal outer tube at a proximal waist distal of the plurality of deflectable fins and at a distal waist proximal of the distal tip member.

2. The thermal ablation device of claim 1, wherein the sealing balloon includes a first cuff, a second cuff, and a central portion extending from the first cuff to the second cuff in the deflated configuration.

3. The thermal ablation device of claim 2, wherein the second cuff is spaced axially apart from the first cuff in the deflated configuration by the central portion of the sealing balloon.

4. The thermal ablation device of claim 2, wherein the first cuff defines a generally annular shape in the deflated configuration.

5. The thermal ablation device of claim 2, wherein the second cuff defines a generally annular shape in the deflated configuration.

6. The thermal ablation device of claim 2, wherein in the inflated configuration of the sealing balloon, the central portion of the sealing balloon defines a radially outermost extent of the sealing balloon.

7. The thermal ablation device of claim 2, wherein in the deflated configuration of the sealing balloon, the first cuff and the second cuff each extend radially outward a greater distance from the distal outer tube than the central portion of the sealing balloon.

8. The thermal ablation device of claim 1, wherein the proximal portion includes:
a proximal inner tube disposed radially outward of the elongate shaft and fixedly attached to the connector,
a proximal middle tube disposed radially outward of the proximal inner tube and spaced apart proximally from the connector, and
a proximal outer tube disposed radially outward of the proximal middle tube and fixedly attached to the connector.

9. The thermal ablation device of claim 8, further including an inflation lumen in fluid communication with the sealing balloon, wherein at least a portion of the inflation lumen is defined by the proximal middle tube and the proximal outer tube.

10. The thermal ablation device of claim 9, wherein at least a portion of the inflation lumen is defined by a recess formed in a wall of the proximal outer tube or a recess formed in a wall of the proximal middle tube.

11. The thermal ablation device of claim 10, wherein an outer surface of the proximal middle tube is in contact with an inner surface of the proximal outer tube.

12. The thermal ablation device of claim 9, wherein the proximal middle tube is radially spaced apart from the proximal inner tube by a first distance and the proximal middle tube is radially spaced apart from the proximal outer tube by a second distance.

13. The thermal ablation device of claim 12, wherein the proximal portion further includes:
a first O-ring disposed between the proximal inner tube and the proximal middle tube; and
a second O-ring disposed between the proximal inner tube and the proximal middle tube;
wherein the proximal inner tube, the first O-ring, the second O-ring, and the proximal middle tube define an insulating air gap extending along a majority of the proximal portion.

14. The thermal ablation device of claim 12, wherein the first distance is greater than the second distance.

15. A thermal ablation device, comprising:
a proximal portion including a handpiece configured to connect to a fluid management system and an inflation port disposed distal of the handpiece;
a distal portion sized and configured for insertion through a cervical opening;
a connector fixedly securing the proximal portion to the distal portion; and
an elongate shaft extending within the proximal portion, through the connector, and within the distal portion, the elongate shaft defining a working lumen;
wherein the distal portion includes:
a distal inner tube disposed radially outward of the elongate shaft and fixedly attached to the connector,
a distal outer tube disposed radially outward of the distal inner tube,
a distal tip member fixedly attached to a distal end of the distal inner tube, a distal end of the distal outer tube, and a distal end of the elongate shaft,
a sealing sleeve fixedly attached to an outer surface of the distal outer tube, the sealing sleeve including a plurality of deflectable fins extending radially outward from the sealing sleeve, and
a sealing balloon in fluid communication with the inflation port and configured to shift between a deflated configuration and an inflated configuration, the sealing balloon being fixedly attached to the distal outer tube at a proximal waist distal of the plurality of deflectable fins and at a distal waist proximal of the distal tip member.

16. The thermal ablation device of claim 15, wherein the sealing balloon is monolithically formed with the plurality of deflectable fins.

17. The thermal ablation device of claim 15, wherein the sealing balloon is fixedly attached to the distal outer tube independently of the plurality of deflectable fins.

18. The thermal ablation device of claim 15, wherein the sealing balloon has a fluid capacity of less than 5 milliliters of inflation fluid.

19. The thermal ablation device of claim 15, wherein:
in the deflated configuration, the sealing balloon defines a first chamber and a second chamber spaced apart from the first chamber; and
in the inflated configuration, the sealing balloon defines a single interior chamber.

20. A thermal ablation device, comprising:
a proximal portion having a first outer diameter;
a distal portion having a second outer diameter less than the first outer diameter; and
a connector disposed between the proximal portion and the distal portion, wherein an outer surface of the connector is tapered from the first outer diameter to the second outer diameter;
wherein the distal portion includes:
a distal inner tube fixedly attached to the connector and a distal tip member,
a distal outer tube disposed radially outward of the distal inner tube and fixedly attached to the connector and the distal tip member,
a sealing sleeve fixedly attached to an outer surface of the distal outer tube, the sealing sleeve including a plurality of deflectable fins monolithically formed therewith and extending radially outward from the sealing sleeve, and
a sealing balloon configured to shift between a deflated configuration and an inflated configuration, the sealing balloon being fixedly attached to the distal outer tube at a proximal waist distal of the plurality of deflectable fins and at a distal waist proximal of the distal tip member.
